(19) United States
(12) Patent Application Publication
(10) Pub. No.: US 2011/0124976 A1
(43) Pub. Date: May 26, 2011

(54) MODEL ENHANCED IMAGING

(75) Inventors: Joerg Sabczynski, Norderstedt (DE); Steffen Renisch, Hamburg (DE); Ingwer-Curt Carlsen, Hamburg (DE); Sven Kabus, Hamburg (DE); Roland Opfer, Hamburg (DE); Michael Kaus, Madison, WI (US); Karl Antonin Bzdusek, Fitchburg, WI (US); Juergen Weese, Aachen (DE); Vladimir Pekar, Toronto (CA)

(73) Assignee: KONINKLIJKE PHILIPS ELECTRONICS N.V., EINDHOVEN (NL)

(21) Appl. No.: 13/055,792

(22) PCT Filed: Jul. 22, 2009
(86) PCT No.: PCT/IB09/53192

§ 371 Date: Jan. 25, 2011

Related U.S. Application Data
(60) Provisional application No. 61/089,061, filed on Aug. 15, 2008.
(60) Provisional application No. 61/113,253, filed on Nov. 11, 2008.
(60) Provisional application No. 61/141,014, filed on Dec. 29, 2008.

(51) Int. Cl. A61B 5/00; G06G 7/60

(52) U.S. Cl. 600/300; 703/11

(57) ABSTRACT

A therapy treatment response simulator includes a modeler (202) that generates a model of a structure of an object or subject based on information about the object or subject and a predictor (204) that generates a prediction indicative of how the structure is likely to respond to treatment based on the model and a therapy treatment plan. In another aspect, a system includes performing a patient state determining in silico simulation for a patient using a candidate set of parameters corresponding to another patient and producing a first signal indicative of a predicted state of the patient, and generating a second signal indicative of whether the candidate set of parameters are suitable for the patient based on a known state of the patient.

The following generally relates to imaging and finds particular application with positron emission tomography (PET); however, it also amenable to other medical imaging and non-medical imaging applications.

Tumors are often treated after diagnosis by radiation therapy. In radiation therapy, a radiation dose high enough to kill tumor cells is delivered to the tumor. Conventional radiation therapy systems such as an intensity modulated radiation therapy (IMRT) system allow precise delivery of a prescribed dose to the target area, spare “normal” tissue surrounding the target area, and “normal” tissue at an increased risk of radiation damage. Usually, the radiation dose is given over weeks in many fractions according to a prescribed fractionation schedule.

Functional imaging can be used to image glucose uptake in living tissue including tumors, which generally exhibit an increased metabolic rate relative to “normal” tissue. With tumors, functional imaging can be used to locate, stage, and monitor growth. An example of such a functional procedure includes ¹⁸F-fluorodeoxyglucose (FDG). With this procedure, the tracer FDG is introduced into the object or subject to be scanned. As the radiopharmaceutical decays, positrons are generated. When a positron interacts with an electron in a positron annihilation event, a coincident pair of 511 keV gamma rays is generated. The gamma rays travel in opposite directions along a line of response, and a gamma ray pair detected within a coincidence time window is recorded as an annihilation event. The events acquired during a scan are reconstructed to produce image or other data indicative of the distribution of the radionuclide and, hence, the distribution of glucose uptake by tissue and tumor.

Functional imaging can also be used to monitor the response of the tumor and the tissue at risk to the radiation from radiation treatment. However, one of the reactions of tissue to the applied radiation is cell death and inflammation due macrophages attracted to the treated site to process or remove the cells killed by the radiation. This processing may lead to increased glucose uptake in radiated tissue. Unfortunately, with functional PET the inflammation-induced increased glucose uptake is not distinguishable from increased glucose uptake in the tumor. As a consequence, the tumor's response to the radiation treatment alone cannot be measured quantitatively by functional PET once the inflammation reaction has started. Rather, the image data shows glucose uptake of both the tumor and the macrophages.

Procedures such as CT, MRI or other imaging procedures that show morphological changes, such as tumor size, can be performed weeks after treatment after the body has had time to respond to the dead cells in order to determine whether a treated tumor has shrunk or grown. Unfortunately, such information does not provide quantitative information and cannot be used to validate the current treatment parameters, assist with changing the parameters, or determine to terminate the treatment until weeks later. In another approach, the effects of the treatment are assumed based on historical data indicative of how others have responded to the treatment. Unfortunately, similar tumors do not necessarily respond the same, lending this approach susceptible to error.

Although, as mentioned above, tumors have been treated via radiation therapy, other treatment regimes have also been used to treat tumors. Unfortunately, treatment decisions are often difficult to make since an individual patient with a tumor often does not respond as expected to the treatment and the treatment may produce undesired side effects. Therefore, the patient is usually monitored during the treatment by additional examinations, e.g., imaging, blood tests, etc. If treatment monitoring shows that the treatment does not produce the expected results, the treatment can be terminated and/or changed. In principle, it may be possible to simulate the development and treatment response of a tumor with the help of a computer model. However, this may be difficult in clinical practice and can be computationally intensive. Moreover, such models rely on demographic data as inputs, which may not be representative of the individual patient.

Forward and inverse planning are the two concepts of linac parameter optimization for external beam radiation therapy. In forward planning, linac parameters such as number of beams and their angular position are manually varied by the user until treatment design parameters, e.g. dose to the target and max dose to normal tissue, are met. IMRT generally cannot be addressed by forward planning due to the number of parameters. Inverse planning is intended to automate the parameter optimization through computational approaches in which optimization of most of the parameters is done algorithmically, but some initial settings like the number of beams, angular positions, and dose-volume or biological objectives and constraints are still manually determined.

Depending on the complexity of the treatment, several iterations of optimization, result review and input parameter adjustment may be required to achieve a clinically acceptable plan. One approach to further automate this iterative process is to compute many possible IMRT solutions by varying inverse planning input parameters in a given interval, and subsequently allowing the user to navigate through the plans and select a plan. However, this approach may be computationally intensive, and requires navigating high-dimensional spaces, which makes it less user-friendly. In addition, various input parameters still need to be specified.

Aspects of the present application address the above-referenced matters and others.

According to one aspect, a therapy treatment response simulator includes a modeler that generates a model of a structure of an object or subject based on information about the object or subject and a predictor that generates a prediction indicative of how the structure is likely to respond to treatment based on the model and a therapy treatment plan.

In another aspect, a therapy system includes a treatment response simulator that generates a parameter map that includes quantitative information indicative of how a first structure of an object or subject is likely to respond to treatment based on a model of the object or subject and a therapy treatment plan for the object or subject and a treatment monitoring system that enhances image data generated from data acquired after the treatment based on the parameter map.

In another aspect, a method includes generating a model indicative of a first structure of an object or subject based on image data indicative of the structure generated from data acquired prior to treatment, generating a prediction indicative of how the first structure is likely to respond to the treatment based on the model and a therapy treatment plan, and generating a parameter map that includes quantitative information about the first structure based on the prediction.

In another aspect, a method includes simulating a first response of a target tissue to a treatment, simulating a second response of a reference tissue to the treatment, treating the target tissue and the reference tissue, determining a third response of the target tissue to the treatment, determining a fourth response of the reference tissue to the treatment, and normalizing the third response based on the fourth response.

In another aspect, a method includes obtaining pre-treatment information, developing a model of a likely affect of a therapy based on the pre-treatment information, obtaining a post-treatment functional image, and comparing the post-treatment functional image to the model to determine the therapy efficacy.

In another aspect, a system includes a processing component that processes patient data corresponding to a patient and a candidate parameter selector that selects a candidate set of simulation parameters for a treatment determining in silico simulation for the patient based on the processed data. A patient state simulator performs a patient state determining in silico simulation for the patient using the candidate set of parameters and produces a first signal indicative of a predicted state of the patient based on the simulation. A decision component generates a second signal indicative of whether the candidate set of parameters are suitable based on the predicted state and a known state of the patient.

In another aspect, a method includes selecting a set of parameters based on processed patient data for a first patient, wherein the set of parameters corresponds to a different patient, performing a first in silico simulation based on the set of parameters, wherein simulation results predict a state of the first patient.

In another aspect, a method includes performing an in silico treatment simulation for a patient based on a set of patient specific parameters for the patient that are determined through an in silico parameter simulation in which the set of patient specific parameters are initially unknown and determined based on known parameters and states of another patient.

Still further aspects of the present invention will be appreciated to those of ordinary skill in the art upon reading and understand the following detailed description.

The invention may take form in various components and arrangements of components, and in various steps and arrangements of steps. The drawings are only for purposes of illustrating the preferred embodiments and are not to be construed as limiting the invention.

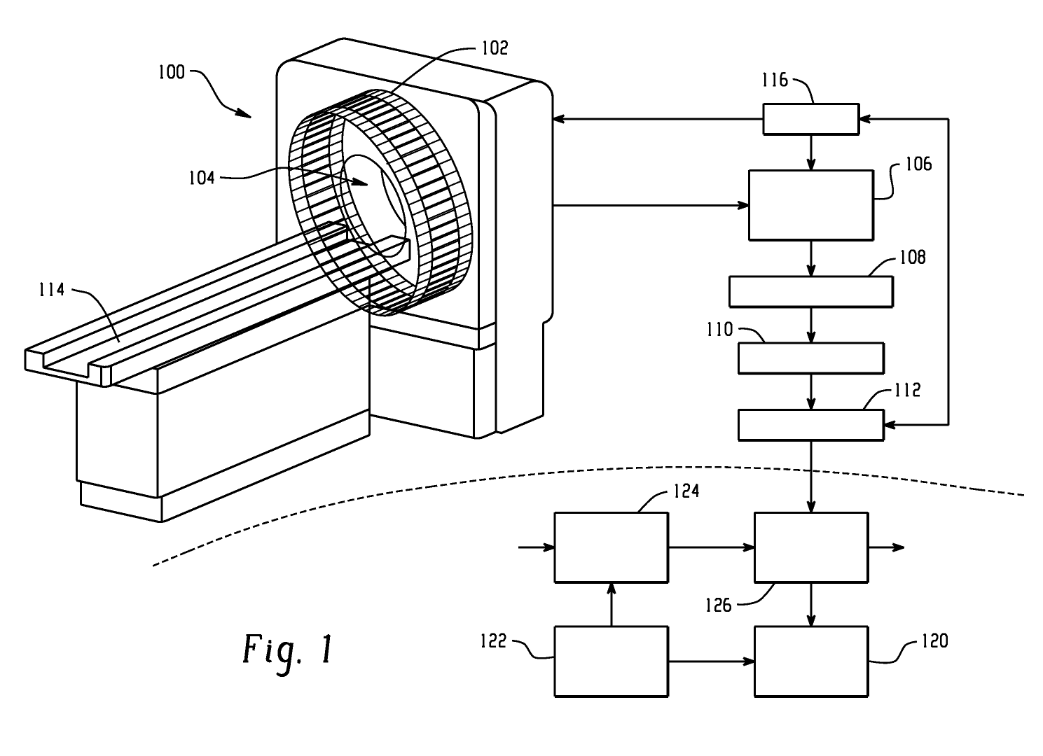

FIG. 1 illustrates an exemplary medical imaging system.

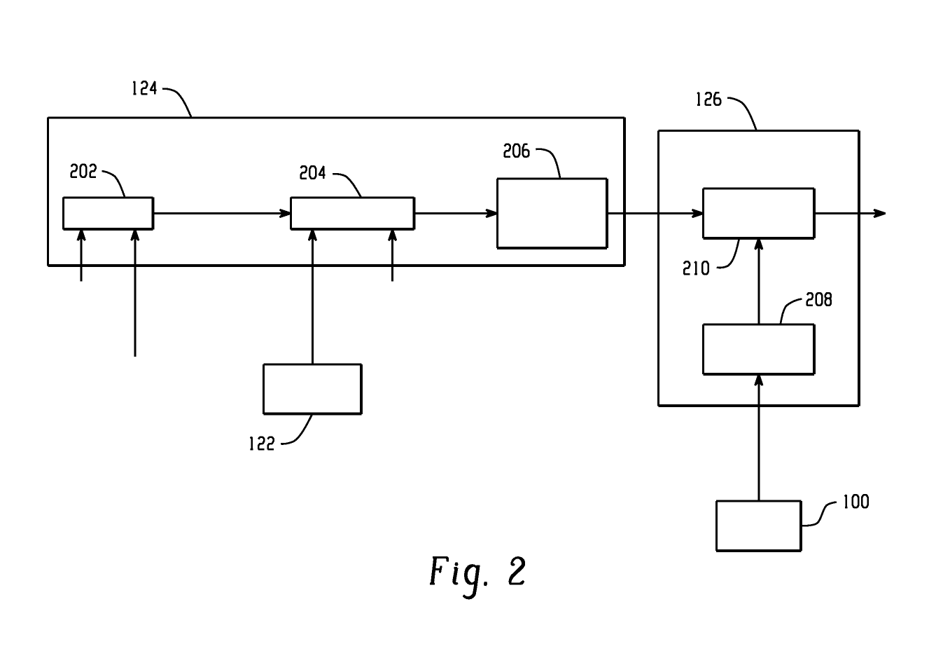

FIG. 2 illustrates an example treatment response simulator and an example treatment monitoring system.

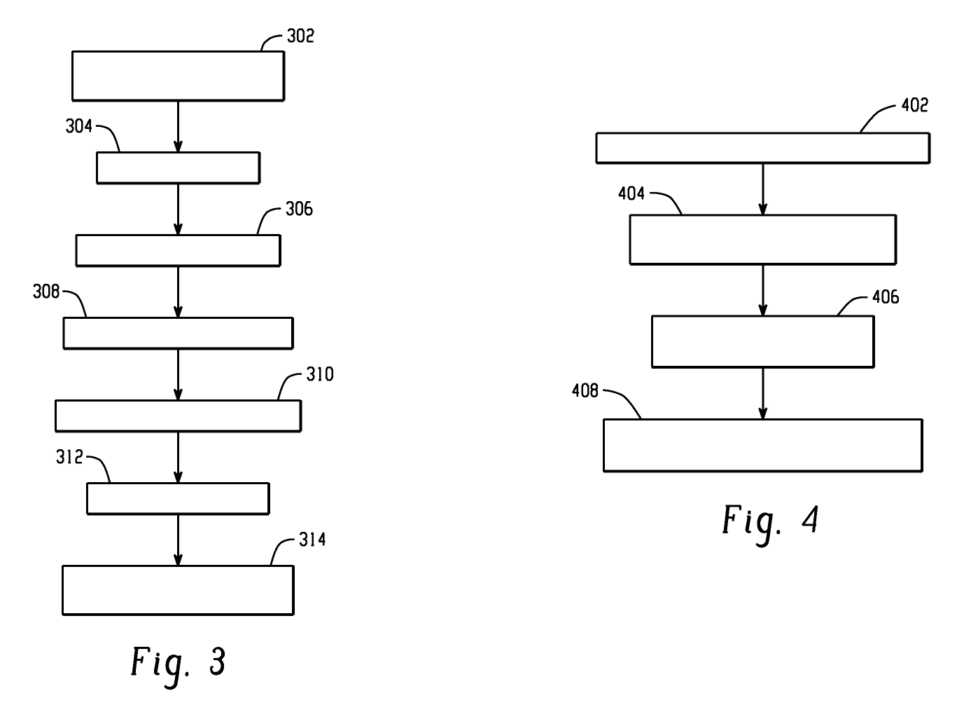

FIGS. 3 and 4 illustrate a method.

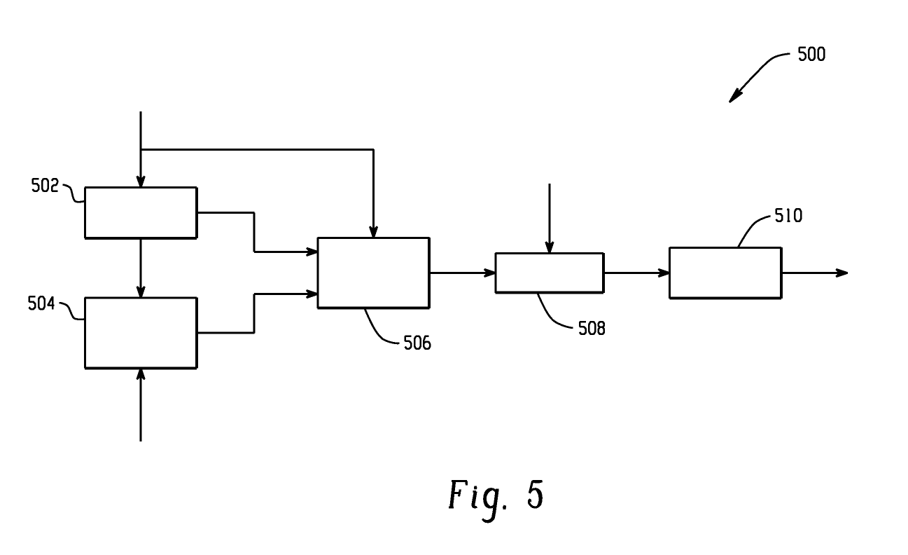

FIG. 5 illustrates an example parameter determiner.

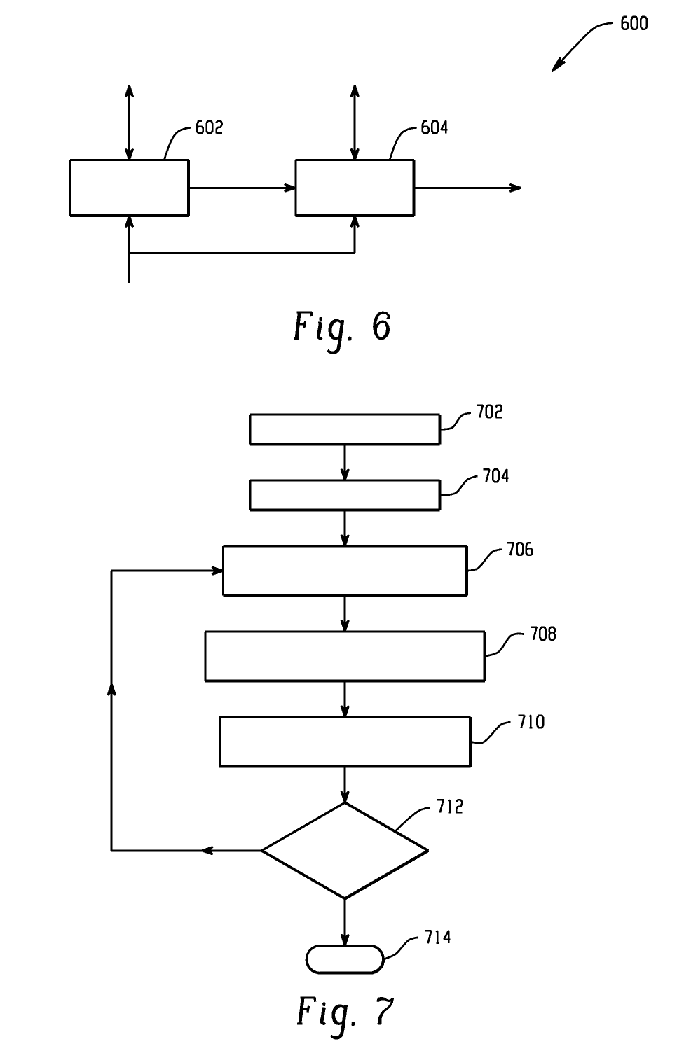

FIG. 6 illustrates an example treatment simulator that employs parameters determined via the parameter determiner of FIG. 5.

FIG. 7 illustrates a method for determining treatment simulation patient specific input parameters via an in silico simulation.

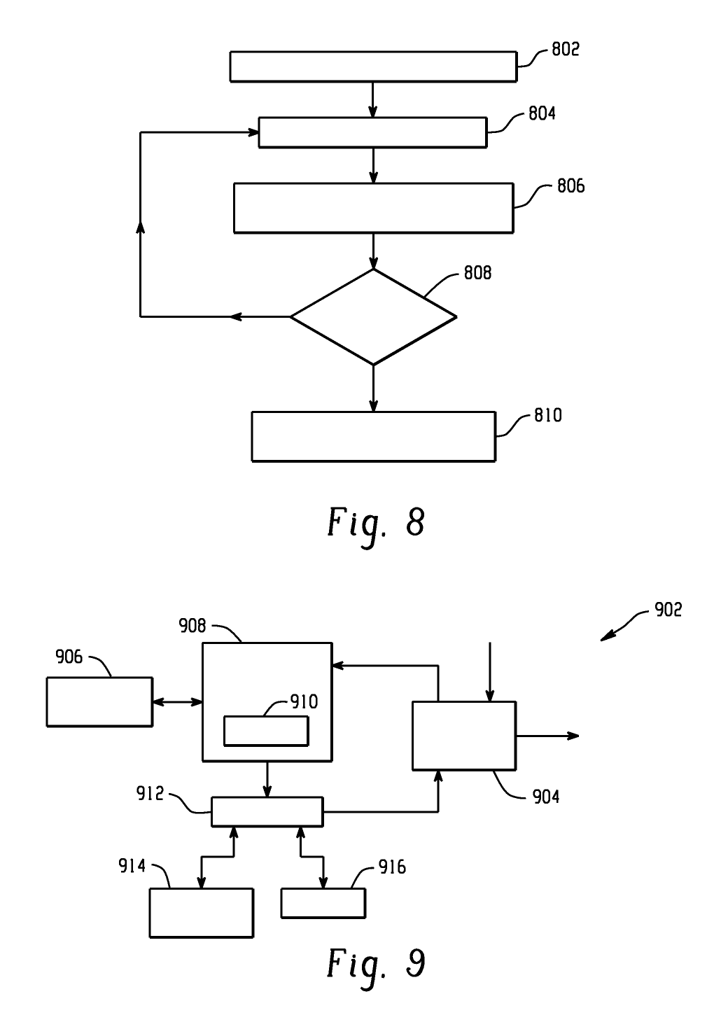

FIG. 8 illustrates a method for employing the treatment simulation patient specific input parameters to perform an in silico treatment simulation.

FIG. 9 illustrates a radiation treatment plan identifier.

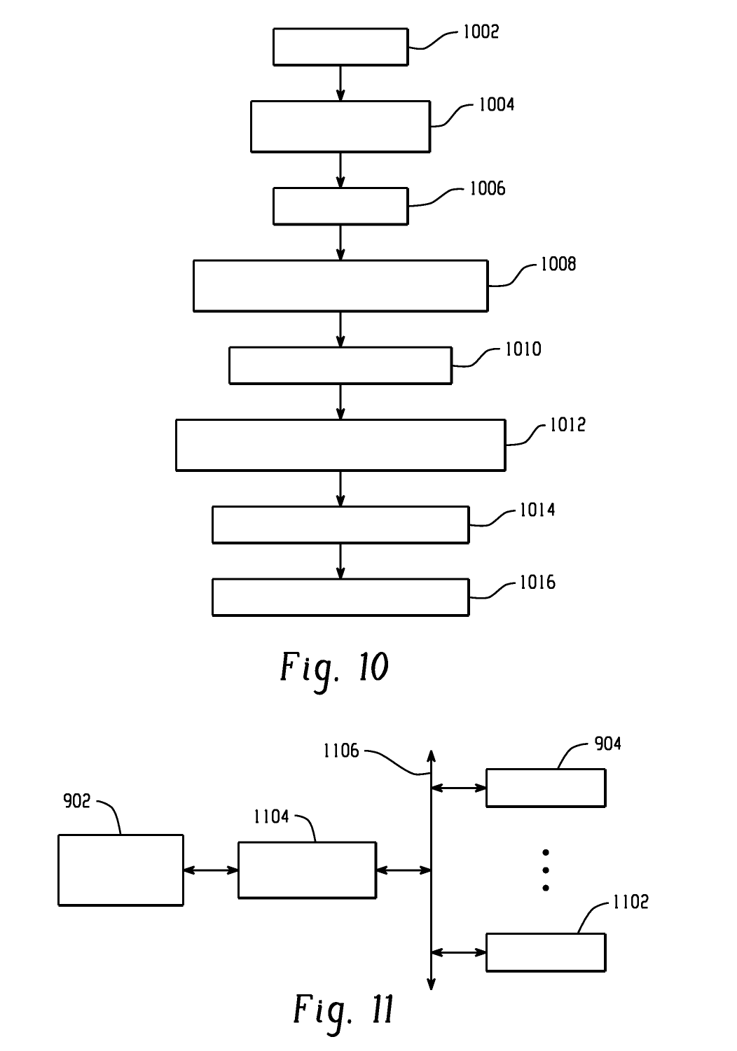

FIG. 10 illustrates a method.

FIG. 11 illustrates radiation treatment plan server.

FIG. 1 illustrates an imaging system 100 that includes gamma radiation sensitive detectors 102 disposed about an examination region 104 along a longitudinal or z-axis in a generally ring-shaped or annular arrangement. In this example, the detectors 102 are arranged in multiple rings along the z-axis. The detectors 102 detect gamma radiation characteristic of positron annihilation events occurring in the examination region 104. A single detector 102 may include one or more scintillation crystals and corresponding photosensors, such as photomultiplier tubes, photodiodes, etc. A crystal produces light when struck by gamma ray, and the light is received by one or more of the photosensors, which generates electrical signals indicative thereof.

A data acquisition system 106 processes the signals and produces projection data such as a list of annihilation events detected by the detectors 102 during image acquisition. List mode projection data typically includes a list of the detected events, with an entry in the list including information such as a time at which the event was detected. A pair identifier 108 identifies pairs of substantially simultaneous or coincident gamma ray detections belonging to corresponding electron-positron annihilation events, for example, via energy windowing (e.g., discarding events outside of an energy rage about 511 keV), coincidence-detecting (e.g., discarding event pairs temporally separated from each other by greater than a threshold), or otherwise.

A line of response (LOR) processor 110 processes the spatial information for each pair of events to identify a spatial LOR connecting the two gamma ray detections. When configured with time of flight (TOF) capabilities, a TOF processor analyzes the time difference between the times of each event of the coincident pair to localize or estimate the position of the positron-electron annihilation event along the LOR. Alternately, the acquired data may be sorted or binned into sinogram or projection bins. The result, accumulated for a large number of positron-electron annihilation events, includes projection data that is indicative of the distribution of the radionuclide in the object.

A reconstructor 112 reconstructs the projection data to generate image data using a suitable reconstruction algorithm such as filtered backprojection, iterative backprojection with correction, etc. A support 114 supports an object or subject to be imaged such as human patient. The object support 114 is movable in coordination with operation of the system 100 for positioning a patient or an imaging subject in the imaging region. A console 116 includes a human readable output device such as a monitor or display and input devices such as a keyboard and mouse. Software resident on the console 116 allows the operator to interact with the scanner 100.

In the illustrated example, the imaging system 100 is used in connection with a therapy treatment system, which may include a radiation therapy, chemotherapy system, a particle (e.g., proton) therapy, a high intensity focused ultrasound (HIFU), an ablation, a combination thereof and/or other treatment system. A treatment planning system 122 is used to generate treatment plans for the therapy treatment system 120. In one instance, the treatment planning system 122 uses image data such as CT, MR, and/or other image data when generating a treatment plan. Such image data may include information such as information that correlates with the electron density of the scanned structured, which can be used to calculate the dose to be imparted by the therapy treatment system 120 to the target region.

A treatment response simulator 124 simulates the response and/or development of treated and/or untreated structures to be treated in the object or subject and generates a prediction indicative of how one or more of the different structures are likely to respond and/or develop with and/or without treatment. As described in greater detail below, the response simulator 124 may generate one or more models based on information such as image data acquired prior to treatment and/or other information about the object or subject, and the one or more models may be used along with treatment information such as the treatment plan and/or object or subject information to generate the prediction. The prediction may be represented in the form of a parameter map for a structure of interest that provides quantitative information about the response. In one instance, the model, prediction, and/or parameter map is generated in silico, or derived by a computer or based computer simulation. An example of a suitable in silico model can be found Stamatakos, et al., “In Silico Radiation Oncology: Combing Novel Simulation Algorithms with Current Visualization Techniques,” Proc IEEE, Vol. 90, No. 11, pp. 1764-1771 (2002). In another instance, the model may additionally or alternatively be empirically and/or theoretically determined.

A treatment monitoring system 126 can be used to monitor the development of treated and/or untreated structure within a scanned region of interest of the object or subject. As described in greater detail below, the monitoring system 126 can determine a response of the different structure to the treatment based on image data from one or more scans such as functional or other scans performed after treatment and the prediction or parameter map of how the one or more different structures are likely to respond to the treatment, which allows for the enhancement (or suppression) of one or more of the structures in the image data. In one instance, this allows independent monitoring of the response of at least two different structures in image data to treatment where the response of the at least two different structure may otherwise be indistinguishable in the image data.

By way of non-limiting example, with a functional scan such as a FDG-PET scan, the different structures may be different tissue in a human patient such as treated and/or untreated tumor cells, macrophages processing the cells killed by the treatment and normal living cells, and the stimulus may relate to radiation, chemo or other therapy used to treat the tumor cells. In such an instance, tracer or glucose uptake identifiable in the functional image data may be from tumor cells and/or the macrophages processing the cells killed by the treatment (e.g., treatment induced inflammation). However, the uptake may not be distinguishable between the tumor cells and macrophages in the image data. The prediction generated by the response simulator 124 may describe how the tumor cells are likely to respond to the radiation or chemo treatment, how normal cells receiving the radiation or chemo are likely to respond, and how tumor cells and/or normal cells receiving none of the treatment are likely to develop. From at least a sub-portion of this information a parameter map(s) including quantitative information indicative of tracer uptake of one or more particular structures (e.g., tumor cells, macrophages, normal living cell, etc.) can be generated and used to emphasize (or suppress) a structure in image data, generated with data acquired at different moments in time after treatment, based on the time of the treatment and the time the data is acquired. For example, a parameter map quantitatively describing tracer uptake of the inflamed tissue can be used to remove the contribution of tracer uptake from the inflamed tissue from the image data, leaving the tracer uptake from the tumor in the image data, which can be used to determine information about the effectiveness of the therapy.

FIG. 2 illustrates a non-limiting example of the response simulator 124 and the monitoring system 126. As noted above, the one or more models, prediction and/or the parameters maps can be determined in silico via computer simulation and/or otherwise. In the illustrated embodiment, the response simulator 124 includes a modeler 202 that generates the one or more models. As depicted, the model generator 202 generates the one or more models based on various information about the object or subject such as a patient, including, but not limited to, image data from data acquired prior to treatment from one or more imaging modalities such as MRI, CT, SPECT, PET, US, X-ray etc., histological data, patient health, medical history, genetics, laboratory test results (e.g., blood values, etc.), pathological information, and/or other information about the patient.

The illustrated response simulator 124 further includes a predictor 204 that predicts how the structures are likely to develop and/or respond to treatment. In one instance, the prediction is based on the one or more models generated by the modeler 202 and information related to the patient such as the current treatment plan (e.g., timing, dose, fractionation scheme, adjuvant medication, etc.), information about the object or subject, and/or other information. The predictor 204 processes this information and produces an output signal that is indicative of how one or more of the structures of interest are likely to respond to the treatment. A parameter map generator 206 generates one or more parameter maps with information indicative of how each of a plurality of different structures are likely to respond to the treatment. In one instance, an individual parameter map is generated for each structure and includes quantitative information about how the corresponding structure is likely to respond.

The monitoring system 126 includes an image data processor 208 that processes image data such as image data corresponding to a time series of functional imaging data generated from data acquired after the treatment, and a data enhancer 210 that enhances the processed image data based on the parameter map. For instance, in order to monitor the treatment response, the system 100 can be used to generate dynamic functional image data at certain points in time after beginning of the treatment. From this image data, the image data processor 208 may derive quantitative information about tracer uptake by different structures. The image data enhancer 210 can enhance this data for a particular structure by subtracting quantitative tracer uptake information based on a parameter map for a different structure that may otherwise be indistinguishable in the image data. The remaining tracer uptake in the image data shows the reaction of the structure of interest to the treatment, and the development of untreated structure of interest. Such information can be used to determine information about the effectiveness of the therapy.

Variations, alternatives and/or other embodiments are discussed.

Although the above is generally described in the context of (treated and untreated) tumor cells, normal cells killed by the treatment, and normal living cells, it is to be appreciated the techniques described herein can be used to discriminate between other structures in a scanned region of interest of an object or subject where the response of different structures to a known stimulus cannot be distinguished in image data from a functional imaging scan. The approach described herein can also be used with other imaging systems and corresponding agents.

FDG-PET is used in an above non-limiting example. However, it is to be understood that other tracers are also contemplated. For example, other suitable tracers include, but are not limited to, other tracers including fluorine-18 such as ¹⁸F-fluorothymidine (FLT), ¹⁸F-fluorothyltyrosine (FET), ¹⁸F-fluoromisonidazole (FMISO), and ¹⁸F-fluoroazomycinarabinofuranoside (FAZA), and/or other tracers with or without fluorine-18.

Although the treatment system 120, the planning system 122, the response simulator 124, and the monitoring system 126 are shown as individual systems, it is to be understood that one or more of these components may be part of the same system.

In another embodiment, the model additionally or alternatively provides qualitative values of the tracer uptake in the different tissue types. In this case, a normal reference tissue volume can be selected. The reference tissue volume should have similar properties as the tumor volume and should receive a similar treatment, for example, radiation dose, fractionation, etc. The simulation is performed for both, the tumor tissue and the reference tissue. Then, a functional scan for therapy monitoring is performed. The resulting prediction is compared to the functional image data for both tissue types. The result for the reference tissue, which shows no increased tracer uptake due to tumor metabolism, is used to normalize the prediction of the inflammation related signal in the tumor. As such, the tumor related tracer uptake can be more accurately determined.

FIG. 3 illustrates a method. It is to be appreciated that the below acts are not limiting and more or less acts and a different ordering of the acts may be used in other embodiments. At 302, pre-treatment information about an object or subject to be treated is obtained. As noted above, such information may include image data and/or other information. At 304, 306 and 308 a model, a prediction and a parameter map that describes how a structure of interest is likely to respond to treatment are respectively generated as described herein, for example, in silico. At 310, the object or subject is treated. At 312, the treated object or subject is imaged via a functional imaging procedure. At 314, the parameter map is used to enhance the response of a treated structure of interest in the image data generated from the functional procedure. The enhanced image data can be used to determine information about the effectiveness of the therapy.

FIG. 4 illustrates a method for predicting the expected efficacy of therapy. At 402, pre-treatment information is obtained. As noted above, this may include imaging as well as other information about the object or subject to be treated. At 404, a model of a likely affect of a therapy is developed based on the pre-treatment information. At 406, post-treatment information such as a functional image is obtained. At 408, the post-treatment information functional image data is compared with the model to determine the efficacy of the therapy. Such information may be displayed and/or otherwise presented, for example, in the form of an image overlay. As noted above, the treatment may include radiation, particle, high intensity focused ultrasound, chemo and/or ablation therapy.

The above embodiments included aspects related to in silico based simulations using known input parameters. The following embodiments involve determining and/or using input parameters for applications where such parameters are not known.

FIG. 5 illustrates a parameter determiner 500 for determining patient specific parameters for an in silico based treatment simulation. The parameter determiner 500 can be part of a stand alone computer such as a workstation, a desktop computer, a laptop, etc., the console 116 or a console of another imaging system, a distributed computing system, etc.

The parameter determiner 500 includes a processing component 502, which processes data. Suitable data includes, but is not limited to, imaging and/or non-imaging data such as diagnostic data captured before treatment, lab tests, patient history, therapy monitoring data captured during or after the treatment, images, image data, and/or other data. Such data can be obtained from sources such as a HIS, RIS, PACS, etc. system, a storage component such as a hard drive, portable memory, etc., a database, a server, a electronic medical record, manually entered, and/or the console 116, another imaging system, and otherwise obtained.

Suitable processing includes, but is not limited to, extracting, deriving, estimating, etc. information from such data. With image based data, the processing may include segmentation, quantification, registration, and/or other information extraction. A candidate parameter selector 504 selects a set of candidate parameters based on the processed data. The set of parameters includes candidate parameters for the in silico treatment simulation. Such parameters may include, but are not limited to information such as initial tumor shape, patient anatomy, physiological values etc, and/or other information.

The selected set of parameters can be obtained from various sources including, but not limited to, a database, a server, an archiver or the like, which stores information from clinical studies, practice, etc. Such information can include information obtained from in silico analyses such as boundary conditions and/or starting values, response to a treatment, etc. Such information may include, but is not limited to, image data, tumor boundaries, clinical symptoms, blood tests, etc. It is to be appreciated that such parameters are known for each of the patients in the clinical study, and at least one of the parameters may be related to progression of a disease and/or a response to a treatment, and may represent a “typical” value for a certain class of patient.

A patient state simulator 506 simulates a known state of the patient based on a selected set of parameters, the patient data, the processed data, and/or other information.

An analyzer 508 analyzes the simulation. This may include comparing simulated results, which predict the current state of the patient based on the input data, with a known state of the patient. The analyzer 508 generates a signal indicative of the comparison. Such information may include a similarity measure or metric such as a metric indicative of a difference or correlation value between the predicted state and the known state. In another embodiment, the analyzer 508 is omitted and a clinician analyzes the simulation.

A decision component 510 determines whether the selected set of parameters is suitable based on the known state of the patient. For example, in one instance the decision component 510 presents the analysis and receives user input as to whether the set of parameters is suitable. In another instance, an automatic or semi-automatic approach is employed. For example, the decision component 510 may compare and/or present the difference or correlation values with a predetermined similarity threshold. Such information can be used by a clinician and/or an executing decision algorithm. The selected set of parameters, or the simulation parameter set, can be stored, presented, and/or otherwise used. In one instance, the set of parameters that renders the simulation results that are closest to the known state of the patient or other parameters is selected as the simulation parameter set.

If more than one set of parameters is available, if the simulation results are deemed not suitable, and/or otherwise, then another simulation can be ran with one or more different sets of parameters. As such, an iterative technique can be used to select the simulation parameter set. In addition, if none of the selected sets of parameters lead to a suitable set of parameters after pre-determined stop criteria, such as a lapse of time, a number of simulations, user termination, etc., the user can determine to use one of the rejected sets and/or otherwise obtain a set of parameters.

FIG. 6 illustrates a treatment determining apparatus 600 that can employ the simulation parameter set and/or other parameters to facilitate determining a treatment and/or a group of suitable treatments.

A treatment selector 602 provides simulation information for various types of treatments. In one instance, the treatment selector 602 selects a treatment based on a state of the patient. For purposes of a simulation, the state may be defined by the parameters of the model. The treatment information can be obtained from a treatment information database, server and/or other information source.

A treatment simulator 604 performs an in silico treatment simulation using the simulation parameter set and the treatment information. In one instance, this includes performing an in silico simulation to predict a future state of the patient based on the current state of the patient, the in silico model, the selected model parameters, and the selected treatment.

In one instance, the treatment simulator 604 presents the simulation results to a user, wherein the user can determine from the simulation whether the treatment is suitable or not based on the simulation. In another instance, an automatic or semi-automatic approach can be used to facilitate the user with making this decision. The results can be stored and/or otherwise used.

If more than one treatment is available, another simulation can be ran for a different treatment. The user can then make a treatment decision based on multiple treatment simulation results for different treatments.

FIG. 7 illustrates a method for determining patient specific in silico based simulation parameters.

At 702, patient data is loaded. Such data can include imaging and/or non-imaging data, obtained from various sources as discussed herein.

At 704, the data is preprocessed. As noted above, this may include segmenting a tumor and/or normal tissue in image data, determining an activity level from functional images, etc. Optionally, this preprocessing may include manual and/or iterative interaction with the data sets.

At 706, one or more sets of parameters or initial conditions for the patient are selected based on the preprocessed data. As noted above, this includes selecting at least one parameter set with known initial conditions corresponding to a different patient(s).

At 708, an in silico simulation is performed with a selected set of parameters to predict a state of the patient.

At 710, the simulation results are analyzed based on the patient data, including the known state of the patient.

At 712, it is determined whether another simulation is to be performed. This can be achieved through manual and/or automatic techniques. If so, then acts 706-712 are repeated.

If not, then at 714, the process of determining parameters via in silico simulation is terminated. One or more of the sets of parameters and/or analysis results can be stored, presented, and/or otherwise used.

FIG. 8 illustrates a method for employing the patient specific in silico simulation determined parameters.

At 802, a set of in silico determined patient specific initial parameters are loaded. Such parameters can be obtained via the method of FIG. 7 or otherwise.

At 804, a type of treatment is selected based on a state of the patient.

At 806, an in silico treatment simulation is performed to predict a future state of the patient based on the current state of the patient and the selected treatment.

At 808, it is determined if another in silico treatment simulation is to be performed. This may be determined based on the results of the in silico simulation and/or otherwise. If so, acts 804 to 808 are repeated.

If not, then at 810 a treatment for the patient can be selected based on the simulations.

FIG. 9 illustrates a treatment plan identifier 902 in connection with a radiation treatment planner 904. The illustrated treatment plan identifier 902 includes a data repository 906, a treatment plan search engine 908, one or more filters 910, a candidate radiation treatment plan identifier 912, an algorithm bank 914, and a profile 916. In other embodiments, the data repository 906 is separate from the treatment plan identifier 902, but the treatment plan identifier 902 still communicates with the data repository 906.

The data repository 906 includes a database or the like with radiation treatment plan information. Such information may include, but is not limited to, an image data set (two, three, and/or four dimensional), segmented image data of a region(s) of interest, a treatment plan parameter set related to beams (e.g., number, angle, etc.), a dosimetric prescription, a critical structure dose objective, patient descriptive information such as demographical data, outcome data, chemotherapy regiments, optimization parameters based on tumor type, stage, etc., and/or other information.

In one instance, the data repository 906 includes validated radiation treatment plans and/or other information that represents the clinical knowledge of the clinicians that created the radiation treatment plans. This may include information that represents the variability across different disease sites (lung, prostate, breast, head and neck, etc.), treatment design variations (between clinical centers, disease stage, fractionation schemes, etc.), geographical variations (e.g. Asian population vs. European or US population, etc.), and/or other information. Such information may be variously cataloged or catalogable, for example, by target type, affected anatomy, patient age, patient gender, patient race, stage, patient history, genetics, etc.

The search engine 908 searches the data repository 906 based on information from the radiation treatment planner 904. Such information can be provided based on various formats such as DICOM (Digital Imaging and Communications in Medicine) and/or other formats. The information provided to the search engine 908 can include data selected by a user of the radiation treatment planner 904 and/or through a default or user defined profile based on the available information.

Such information may include various information such as, but not limited to, tumor related data (e.g., type, size, stage, etc.), patient data (e.g., age, sex, gender, etc.), image data (e.g., segmented regions of the target tissue, non-target tissue, etc.), treatment information, and/or other information. With this information, the search engine 908 searches the data repository 906 for patients with similar anatomical features, tumor type, treatment information and/or other information.

The illustrated search engine 908 can use various filters 910, concurrently and/or serially, to facilitate the search. For example, a first filter of the filters 910 may be employed to reduce the searchable data based on tumor type. Where the data in the repository 906 is cataloged, this may include locating suitable data by index and/or otherwise. A second filter of the filters 910 can be employed to further reduce the searchable data based on tumor stage.

Third through Nth filters of the filters 910 can be concurrently employed based on the available segmented regions of interest, for example, identifying data sets in which the shape of the anatomy is more similar to the shape of the anatomy of the current patient. It is to be understood that the above description related to filters is provided for explanatory, and in some embodiments, filters are not employed and/or omitted. A user may also manually select data to search and/or data to exclude from the search.

The search results are provided to the candidate radiation treatment plan identifier 912. The identifier 912 identifies one or more radiation treatment plans from the search results. In one instance, the identifier 912 identifies one or more best-fit treatment plans based on an algorithm from an algorithm bank 914. Suitable algorithms include, but are not limited to, algorithms based on image based similarity metrics such as those employed in image registration algorithms such as mutual information, cross-correlation, etc., structure-based similarity metrics, for example, based on a comparison of region of interest characteristics such as volume, shape, geometric constellation, key features of an image that define the size, shape, etc of the patient, and/or other similarity metrics.

Suitable algorithms may also include pattern recognition based algorithms, for example, using multi-dimensional feature vectors for a variety of features extracted from the patient data, including demographics, tumor staging, tumor location, etc. In another embodiment, machine learning algorithms, implicitly or explicitly trained classifiers, Bayesian networks, neural network, cost functions, etc. can additionally or alternatively be employed. Using such algorithms allows for automatically identifying radiation treatments plans based on similarities between the current patient and patients in the database rather than through computationally expensive approaches. Suitable algorithms may also include methods for content-based image retrieval from a database.

In some embodiments, a profile 916 may be used to facilitate plan identification. For example, the profile 916 may include a predetermined minimum and/or maximum number of plans threshold. A minimum number of plans threshold may be used to ensure that at least radiation treatment plan is identified or that the user will have a choice of radiation treatment plans to choose from. A minimum maximum of plans threshold may be used to limit the number of plans the user will have to choose from.

In addition, a similarity threshold may be predetermined The similarity threshold may set an error and/or time threshold at which the selection process terminates, regardless of how many radiation treatment plans have been identified. Furthermore, the selection process and/or results can be previewed or reviewed by a user who can manually terminate the selection process and/or modify selection parameters.

In one instance, the one or more identified radiation treatment plans are provided to the radiation treatment planner 904. Again, data transfer can be based on various formats such as DICOM and/or other formats. The user can interact with the planner 904 to select one of the suggested treatment plans for the patient. The user can also modify one or more of the parameters of the selected plan and/or request that the treatment plan determiner 900 repeat the process using the same or different parameters. Such interaction may be through a graphical user interface (GUI), a command line interface, and/or other interface.

The selected radiation treatment plan can implemented like a conventionally determined plan. For example, the radiation treatment plan can be used to deliver a single dose or fractional dose over a period of time. In addition, the radiation treatment plan can be modified based on patient response, a tumor response, new information, and/or otherwise. Moreover, the treatment plan identifier 902 can be used again with information obtained during treatment and/or otherwise to provide an updated radiation treatment plan based on the new information.

In another instance, a treatment plan mapper 918 maps the selected radiation treatment plan to fit the anatomy and/or other characteristics of the target image. This can be achieved using a variety of methods similar to the search algorithms previously discussed including but not limited too intensity based similarity metrics and pattern based methods. The treatment plan mapper 918 can be part of the treatment plan identifier 902, the radiation treatment planner 904, or a separate component.

The radiation treatment planner 904 can be a computing system such as a workstation, desktop computer, a laptop or the like. As such, the radiation treatment planner 904 can include one or more processors and memory for storing computer executable instructions, data to be processed, data being processed, processed data, and/or other information. The illustrated radiation treatment planner 904 includes computer executable instructions that, when executed by the processor, provide a treatment planning application with functionality such as image display, manual and automated segmentation tools, image fusion tools, three dimensional conformal radiation therapy (3D CRT) planning, inverse IMRT optimization, dose calculation, etc.

The radiation treatment planner 904 obtains various information such as image data, including two, three and/or four dimensional image data. Such image data may represent the anatomy to be treated, including target tissue, non-target or tissue at risk of being affected by the treatment, non-target tissue not at risk, and/or other tissue. Such image data can be obtained via various imaging modalities such as computed tomography (CT) magnetic resonance (MR), single photon emission tomography (SPECT), etc., including a combination or hybrid imaging system such as a CT/MR imaging system.

The radiation treatment planner 904 may receive the image from the imaging system, an archive system such as a HIS, RIS, or PACS system, portable storage, a database, a server, an electronic medical record, manual entry by a human or robot, and/or otherwise. The radiation treatment planner 904 also obtains a treatment type, including radiation therapy, chemotherapy, particle therapy, high intensity focused ultrasound (HIFU), ablation, image guided radiation therapy, and/or other treatment type. Such information can be entered via a user and/or otherwise.

In another embodiment, the treatment identifier 902 can also be used to determine a treatment type. For instance, the search may not indicate a particular radiation treatment type. For example, the clinician may not have determined a treatment type yet or may not be able to at this point in the planning. In such an instance, the identified treatment plans may include different types. In another instance, the treatment identifier 902 could be used to provide information about to optimally treat the patient, e.g. support decision making about modality (3D CRT vs IMRT vs VMAT; EBRT alone vs EBRT+Chemo, etc.)

FIG. 10 illustrates a method.

At 1002, a tumor is staged for a patient is diagnosed with a tumor.

At 1004, treatment alternatives are selected.

At 1006, the tumor is imaged.

At 1008, information about the patient, the treatment, the tumor, and/or other information is obtained. Such information may correspond to image data such as segmented regions of interest, patient data such as demographics, tumor data such as size, shape, stage, type, etc., prior treatment data, other information as described herein, and/or other information.

At 1010, a treatment type is selected. As described herein, in some embodiments a treatment type is not selected yet.

At 1012, the treatment plan identifier 904 is used as described herein or otherwise to identify one or more radiation treatment plans for the patients. As discussed herein, this includes matching various information about the current patient with the patient information in the data repository 906, and identifying candidate radiation treatment plans from the data repository 906 based on the matching.

At 1014, information about the selected one or more treatment plans is presented to a clinician, who can select a plan to be used for the patient. This plan may be mapped to the current patient using methods discussed previously for review. As discussed herein, the clinician may modify treatment plan parameters and/or request the treatment plan identifier 902 to repeat the candidate identification process.

At 1016, one or more treatment plans are selected. In one instance, a plan can be manually selected from the one or more presented plans, and the input parameters of the selected plan can be applied to optimize a new treatment plan. In another instance, several plans can be generated by using the parameters from a plurality of or of all the plans identified in the database, the one or more of the generated plans can be presented to the user, who can select one or more of them.

At 1018, the selected plan is mapped to a treatment plan for the patient to be treated. In one instance, this may include fitting the selected radiation treatment plan to the anatomy and/or other characteristics of the target image as described herein.

In another instance, instead of retrieval of a plan from a data repository 906, a plan can be selected from the data repository 906 and parameters from the selected plan can be used as input for further IMRT optimization. This would allow for increment build up of the data repository 906.

FIG. 11 illustrates another embodiment. In this embodiment, a radiation treatment (RT) client such as the radiation treatment planner 904 and/or at least one other client 1102 communicates with a subscription service or server 1104 via a network 1106. The subscription server 1104 provides a subscription based service in connection with the treatment plan identifier 902. In one instance, the service is Internet based.

By way of example, a health care facility or other facility can subscribe to the subscription server 1104 on fee or other bases. On the basis of the subscription, the subscription server 1104 will process treatment plan requests from the client 904 and/or the at least one other client 1102. Processing such a request may entail employing the treatment plan identifier 902 to identify candidate treatment plans as described herein.

In instances where the candidate treatment plans are modified, the resulting treatment plan may be provided for inclusion into the data repository 906.

The above may be implemented by way of computer readable instructions, which when executed by a computer processor(s), cause the processor(s) to carry out the described techniques. In such a case, the instructions are stored in a computer readable storage medium associated with or otherwise accessible to the relevant computer. The described techniques need not be performed concurrently with the data acquisition.

The invention has been described with reference to various embodiments. Modifications and alterations may occur to others upon reading the detailed description. It is intended that the invention be constructed as including all such modifications and alterations insofar as they come within the scope of the appended claims or the equivalents thereof.

1. A therapy treatment response simulator, comprising:
a modeler that generates a model of a structure of an object or subject to be treated based on information about the object or subject; and
a predictor that generates a predicted response indicative of how the structure is likely to respond to the treatment based on the model and a therapy treatment plan.

2. The simulator of claim 1, further including:
a parameter map generator that generates a parameter map that includes quantitative information indicative of the predicted response.

3. The simulator of claim 2, wherein the quantitative information includes quantitative information indicative of tracer uptake related to the structure.

4. The simulator of claim 3, wherein the structure includes macrophages processing cells killed by the treatment.

5. (canceled)

6. The simulator of claim 4, wherein the tracer uptake by the macrophages is similar to tracer uptake by a tumor being treated, wherein the macrophages process normal cells around tumor that have been killed by the treatment.

7. The simulator of claim 1, wherein the treatment includes radiation therapy, chemotherapy, particle therapy, high intensity focused ultrasound, ablation or combinations thereof.

8. The simulator of claim 1, wherein the information about the object or subject includes image data generated from data acquired prior to the treatment.

9. The simulator of claim 1, wherein the information about the object or subject includes one or more of histological data, patient health information, medical history, genetics, laboratory test results, or pathological information.

10. The simulator of claim 1, wherein at least one of the model, the prediction or the parameter map is generated in silico.

11. A therapy system, comprising:
a treatment response simulator that generates a parameter map that includes quantitative information indicative of how a first structure of an object or subject is likely to respond to treatment based on a model of the object or subject and a therapy treatment plan for the object or subject; and
a treatment monitoring system that enhances image data generated from data acquired after the treatment based on the parameter map.

12. (canceled)

13. The therapy system of claim 11, wherein the image data includes information indicative of tracer uptake by the first structure and at least one different structure of the object or subject.

14. (canceled)

15. The therapy system of claim 11, wherein the treatment monitoring system includes:
an image data processor that processes the image data to generate quantitative information about two or more structures receiving treatment, wherein one of the two or more treated structures includes the first structure; and
an image data enhancer that enhances a second structure of the two or more structures in the image data by subtracting the quantitative information about the first structure from the image data.

16. The therapy system of claim 11, wherein the treatment response simulator includes:
a modeler that generates the model based on image data generated from data acquired prior to the treatment and the object or subject;
a predictor that generates a prediction indicative of how the first structure is likely to respond to the treatment based on the model and the therapy treatment plan; and
a parameter map generator that generates the parameter map based on the prediction.

17. (canceled)

18. The therapy system of claim 11, wherein the treatment response simulator generates the parameter map based on one or more of histological data, patient health information, medical history, genetics, laboratory test results, or pathological information.

19. A method, comprising:
generating a model indicative of a first structure of an object or subject based on image data indicative of the structure generated from data acquired prior to treatment;
generating a prediction indicative of how the first structure is likely to respond to treatment based on the model and a therapy treatment plan; and
generating a parameter map that includes quantitative information about tracer uptake by the first structure based on the prediction.

20. The method of claim 19, further including:
generating quantitative information about two or more treated structures, wherein one of the two or more treated structures includes the first structure, based on image data generated from an imaging procedure performed after the treatment; and
enhancing a second structure of the two or more treated structures in the image data based on the quantitative information about the first structure.

21. The method of claim 19, further including:
suppressing the quantitative information about the first structure in the image data.

22. The method of claim 19, wherein the image data includes information indicative of tracer uptake.

23. A method, comprising:
simulating a first response of a target tissue to a treatment;
simulating a second response of a reference tissue to the treatment;
treating the target tissue and the reference tissue;
determining a third response of the target tissue to the treatment;
determining a fourth response of the reference tissue to the treatment; and
normalizing the third response based on the fourth response.

24. The method of claim 23, wherein the reference tissue includes similar tracer uptake properties as the target tissue.

25. The method of claim 23, wherein the first and second tissue are treated with one of a substantially similar radiation dose or fractionation.

26. The method of claim 23, wherein the third and fourth response are determined based on a functional scan performed after the treatment.

27. A method of determining therapy efficacy, comprising:
obtaining pre-treatment information;
developing a model of a likely affect of a therapy based on the pre-treatment information;
obtaining a post-treatment functional image data; and
comparing the post-treatment functional image to the model to determine the therapy efficacy.

28. The method of claim 27, further comprising displaying information indicative of the comparison.

29. The method of claim 28, wherein the information is in the form of an image overlay.

30. The method of claim 27, wherein the treatment is one of radiation, particle, high intensity focused ultrasound, chemo or ablation therapy.

31-42. (canceled)

43. A method, comprising:
selecting a set of parameters based on processed patient data for a first patient, wherein the set of parameters corresponds to a different patient; and
performing a first in silico simulation based on the set of parameters, wherein simulation results predict a state of the first patient.

44. The method of claim 43, further including employing the set of parameters to perform a second in silico simulation based on the set of parameters when the predicted state represents a known state of the first patient.

45. The method of claim 44, wherein the second in silico simulation predicts a treatment response of the first patient.

46. The method of claim 44, further including:
comparing a difference value between a first value indicative of the predicted state and a second value indicative of the known state with a predetermined threshold; and
employing the set of parameters to perform a second in silico simulation when the difference is less than the threshold.

47. The method of claim 44, further including:
generating a similarity metric indicative of a similarly between the predicted state and the known state;
comparing the similarity metric with a predetermined threshold; and
employing the set of parameters to perform a second in silico simulation when the similarity metric exceeds the threshold.

48. The method of claim 43, wherein the set of parameters includes known boundary and initial conditions and post treatment parameters corresponding to another patient.

49. (canceled)

50. A system for identifying at least one candidate radiation treatment plan, comprising:
a data repository that includes radiation treatment plans for previously treated patients and related information about the previously treated patients;
a treatment plan search engine that searches the data repository (906) for radiation treatment plans based on information about a patient to be treated and generates search results; and
a candidate radiation treatment plan identifier that identifies at least one of radiation treatment plan in the search results based on a similarity between the information about the patient to be treated and corresponding information about the previously treated patient.

51. The system of claim 50, further including a mapper that maps the identified radiation treatment plan to a radiation treatment plan for the patient to be treated based on at least one of deformable image registration, pattern matching, or parameter matching.

52. The system of claim 50, wherein the information includes image data, and the candidate radiation treatment plan identifier identifies the at least one of radiation treatment plan based on a similarity between the image data.

53. The system of claim 52, wherein the candidate radiation treatment plan identifier identifies the at least one of radiation treatment plan based on a similarity between dimensions of corresponding anatomical structures in the image data.

54. The system of claim 50, wherein the information includes tumor characteristics, and the candidate radiation treatment plan identifier identifies the at least one of radiation treatment plan based on a similarity between the tumor characteristics.

55. The system of claim 50, wherein the information includes data that represents tissue variability across different disease sites.

56. The system of claim 50, wherein the information includes data that represents treatment design variations amongst treatment sources.

57. The system of claim 50, wherein the information includes data that represents patient demographic variations.

58. The system of claim 50, wherein the treatment plan search engine applies a filter to the data repository to select a subset of the radiation treatment plans based on the information about the patient to be treated.

59. The system of claim 58, wherein the filter identifies at least one of tumor characteristics of interest or patient demographics of interest.

60. The system of claim 50, wherein the candidate radiation treatment plan identifier identifies the at least one of radiation treatment plan based on image registration between corresponding regions of interest in segmented images for the patient to be treated and the previously treated patient.

61. The system of claim 50, wherein the candidate radiation treatment plan identifier identifies the at least one radiation treatment plan corresponding to a maximum of a similarity measure between the information.

62-77. (canceled)

78. A method, comprising: identifying a candidate treatment plan for treating a tumor in a first patient based on matching characteristics of the first patient with corresponding characteristics of a previously treated patient, wherein the candidate treatment plan is selected from a repository of validated treatment plans for previously treated patients.

79. The method of claim 78, further comprising mapping parameters of the identified candidate treatment plan to a treatment plan for the first patient.

80. The method of claim 78, further comprising performing an in silico simulation based on the identified candidate treatment plan to predict a treatment response of the first patient to the identified candidate treatment plan.

81. The method of claim 78, further comprising performing an in silico simulation based on the identified candidate treatment plan to predict a current state of the first patient.

82. The method of claim 78, wherein at least on of the validated treatment plans in the repository is determined through an in silico parameter simulation.

83. The method of claim 78, wherein identified candidate treatment plan is identified through an in silico simulation.

84. The method of claim 78, further comprising:
generating a model indicative of a first structure of the first patient; and
generating a prediction indicative of how the first structure is likely to respond to a treatment based on the model and the identified candidate treatment plan.

85. The method of claim 84, further comprising:
obtaining post-treatment data; and
determining an efficacy of the treatment based on a comparison between the post-treatment data and the prediction.

86. The method of claim 78, further comprising:
generating a parameter map that includes quantitative information indicative of how a first structure is likely to respond to a treatment based on a model of the structure and the identified candidate treatment plan.

87. The method of any claim 86, further comprising: enhancing image data of the first structure based on the parameter map.
